(19) United States
(12) Patent Application Publication
(10) Pub. No.: US 2026/0363976 A1
(43) Pub. Date: Nov. 19, 2026

(54) PERSONAL CARE COMPOSITION COMPRISING A VOLATILE ALKANE MIXTURE AND CROSSLINKED ELASTOMERS

(71) Applicant: MOMENTIVE PERFORMANCE MATERIALS INC., Niskayuna, NY (US)

(72) Inventors: Dian LU, Shanghai (CN); Amar PAWAR, Tarrytown, NY (US); Benjamin FALK, Tarrytown, NY (US); Polina TSUPKO, Tarrytown, NY (US); Xu QIN, Tarrytown, NY (US); Mana TAMAMI, Tarrytown, NY (US)

(73) Assignee: MOMENTIVE PERFORMANCE MATERIALS INC., Niskayuna, NY (US)

(21) Appl. No.: 19/135,768

(22) PCT Filed: Dec. 5, 2022
(86) PCT No.: PCT/CN2022/136537

§ 371 Date: Jun. 4, 2025

(51) Int. Cl. A61K 8/31; A61K 8/04; A61K 8/27; A61K 8/891; A61K 8/892; A61Q 17/04; A61Q 19/00; A61Q 19/08

(52) U.S. Cl. CPC A61K 8/31; A61K 8/042; A61K 8/27; A61K 8/891; A61K 8/892; A61Q 17/04; A61Q 19/007; A61Q 19/08; A61K 2800/412; A61K 2800/594; A61K 2800/654

(57) ABSTRACT

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more crosslinked elastomers.

### FIELD OF THE DISCLOSURE

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more crosslinked elastomers. The present disclosure also relates to linear alkanes comprising about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture. The present disclosure further relates to a crosslinked elastomer selected from the group consisting of a crosslinked siloxane elastomer, a crosslinked polyurethane elastomer, a crosslinked polyester elastomer, and combinations thereof.

### BACKGROUND

Swollen crosslinked polymers are used in personal care formulations to provide cushioning and powdery afterfeel without heavy or greasy feel. The afterfeel of swollen crosslinked elastomers is often determined by the properties of solvents used to swell and/or disperse the crosslinked elastomer in. Non-volatile solvents provide moisturized, satin after-feel, while volatile oils provide dry, powdery after-feel. Currently used volatile oils include silicone-based solvents such as cyclopentasiloxane and methyl trimethicone, and hydrocarbon solvents such as isododecane and isohexadecane. The silicone-based solvents present challenges due to non-biodegradibility. The hydrocarbon volatile solvents such as isododecane (IDD) can flash off the skin too quickly leaving the skin dry and uncomfortable. Other ester-based organic solvents or other hydrocarbon solvents, such as isohexadecane, are non-volatile and do not satisfy the need of providing dry, powdery, and non-tacky after-feel. When combining the present disclosure with crosslinked elastomer powders, the personal care composition provides dry, powdery afterfeel without excessive dryness and greasiness.

### BRIEF SUMMARY OF THE DISCLOSURE

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more crosslinked elastomers.

In some aspects, the linear alkanes comprise about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

In some aspects, the crosslinked elastomer is a swellable crosslinked elastomer, a non-swellable crosslinked elastomer, or combinations thereof.

In some aspects, the crosslinked elastomer has particle sizes from about 1 micron to about 500 microns.

In some aspects, the swellable crosslinked elastomer swells with a solvent to form a swollen crosslinked elastomer.

In some aspects, the solvent is about 10% or more by weight relative to the weight of the swollen crosslinked elastomer.

In some aspects, the solvent is a volatile alkane mixture, a silicone solvent, a hydrocarbon solvent, an organic solvent, a fluorinated solvent, or combinations thereof.

In some aspects, the solvent comprises about 0% to about 100% by weight of the volatile alkane mixture relative to the total weight of the solvent.

In some aspects, the swollen crosslinked elastomer is a gel.

In some aspects, the swollen crosslinked elastomer has viscosity of about 10,000 cP to about 2,000,000 cP at 25 °C.

### BRIEF DESCRIPTION OF THE FIGURE

The accompanying FIGURE, which is incorporated herein, forms part of the specification and illustrates embodiments of the present disclosure. Together with the description, the FIGURE further serves to explain the principles of and to enable a person skilled in the relevant art(s) to make and use the disclosed embodiments. The FIGURE is intended to be illustrative, not limiting.

FIG. 1 describes the volatility of Formulation Examples 1a and 1b.

### DETAILED DESCRIPTION

As used above, and throughout the description, the following terms, unless otherwise indicated, shall be understood to have the following meanings.

Unless stated otherwise, the terms “a” and “an” and “the” and similar references used in the context of describing a particular aspect of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” encompasses the range of experimental error that occurs in any measurement.

The term, “hydrocarbon” as used herein refers to any chemical structure containing hydrogen atoms and carbon atoms.

The term “alkyl” means any monovalent, saturated straight chain or branched chain hydrocarbon group; and the term “alkane” means organic compounds consisting entirely of single-bonded carbon and hydrogen atoms.

Representative examples of alkyls include, but are not limited to, methyl, ethyl, propyl and isobutyl. Examples of alkanes include, but are not limited to, methane, ethane, propane and isobutane.

The term “aryl” includes any aromatic hydrocarbon from which one hydrogen atom has been removed. Specific, non-limiting examples of aryl groups include phenyl and naphthalenyl.

The term “linear C₉-C₁₈ alkanes” means linear alkanes having 9 to 18 alkanes. Examples of linear C₉-C₁₈ alkanes include, but are not limited to, n-decane, n-dodecane, and n-tetradecane.

The term “branched C₁₀-C₁₄ alkane” means branched alkanes having 10 to 14 carbon atoms. Examples of branched C₁₀-C₁₄ alkane include, but are not limited to, isodecane and isododecane.

Other than in the working examples or where otherwise indicated, all numbers expressing amounts of materials, reaction conditions, time durations, quantified properties of materials, and so forth, stated in the specification and claims are to be understood as being modified in all instances by the term “about”.

It will be understood that any numerical range recited herein includes all sub-ranges with that range and any combination of the various endpoints of such ranges or sub-ranges.

It will be further understood that any compound, material or substance which is expressly or implicitly disclosed in the specification and/or recited in a claim as belonging to a group of structurally, compositionally and/or functionally related compounds, materials or substances includes individual representatives of the group and all combinations thereof.

### I. Volatile Alkane Mixtures

The present disclosure relates to personal care compositions comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more crosslinked elastomers.

In some aspects, the volatile alkane mixture comprises about 10% to about 90% by weight of linear C₉-C₁₈ alkanes, about 15% to about 90% by weight of linear C₉-C₁₈ alkanes, about 20% to about 90% by weight of linear C₉-C₁₈ alkanes, about 25% to about 85% by weight of linear C₉-C₁₈ alkanes, about 30% to about 85% by weight of linear C₉-C₁₈ alkanes, about 35% to about 80% by weight of linear C₉-C₁₈ alkanes, about 40% to about 80% by weight of linear C₉-C₁₈ alkanes, about 45% to about 75% by weight of linear C₉-C₁₈ alkanes, about 50% to about 75% by weight of linear C₉-C₁₈ alkanes, about 55% to about 70% by weight of linear C₉-C₁₈ alkanes, or about 60% to about 70% by weight of linear C₉-C₁₈ alkanes relative to the total weight of the alkane mixture.

In some aspects, the volatile alkane mixture comprises about 30% to about 85% by weight of linear C₉-C₁₈ alkanes relative to the total weight of the alkane mixture. In some aspects, the volatile alkane mixture comprises about 50% to about 75% by weight of linear C₉-C₁₈ alkanes relative to the total weight of the alkane mixture.

In some aspects, the linear alkanes comprise about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

In some aspects, the linear alkanes comprise about 0% to about 5% by weight of n-decane, about 1% to about 4% by weight of n-decane, or about 2% to about 3% by weight of n-decane relative to the total weight of the alkane mixture. In some aspects, the n-decane is about 0% by weight, about 1% by weight, about 2% by weight, about 3% by weight, about 4% by weight, or about 5% by weight relative to the total weight of the alkane mixture. In some aspects, the n-decane is about 1% by weight relative to the total weight of the alkane mixture. In some aspects, the n-decane is about 5% by weight relative to the total weight of the alkane mixture.

In some aspects, the linear alkanes comprise about 50% to about 80% by weight of n-dodecane, about 55% to about 75% by weight of n-dodecane, about 60% to about 70% by weight of n-dodecane, or about 65% to about 70% by weight of n-dodecane relative to the total weight of the alkane mixture. In some aspects, the n-dodecane is about 50% by weight, about 55% by weight, about 60% by weight, about 65% by weight, about 70% by weight, about 75% by weight, or about 80% by weight relative to the total weight of the alkane mixture. In some aspects, the n-dodecane is about 60% by weight relative to the total weight of the alkane mixture. In some aspects, the n-dodecane is about 65% by weight relative to the total weight of the alkane mixture. In some aspects, the n-dodecane is about 70% by weight relative to the total weight of the alkane mixture.

In some aspects, the linear alkanes comprise about 1% to about 5% by weight of n-tetradecane, about 2% to about 4% by weight of n-tetradecane, or about 3% to about 4% by weight of n-tetradecane relative to the total weight of the alkane mixture. In some aspects, the n-tetradecane is about 1% by weight, about 2% by weight, about 3% by weight, about 4% by weight, or about 5% by weight relative to the total weight of the alkane mixture. In some aspects, the n-tetradecane is about 1% by weight relative to the total weight of the alkane mixture. In some aspects, the n-tetradecane is about 5% by weight relative to the total weight of the alkane mixture.

In some aspects, the linear alkanes comprise about 0% to about 5% by weight of n-hexadecane, about 1% to about 4% by weight of n-hexadecane, or about 2% to about 3% by weight of n-hexadecane relative to the total weight of the alkane mixture. In some aspects, the n-hexadecane is about 0% by weight, about 1% by weight, about 2% by weight, about 3% by weight, about 4% by weight, or about 5% by weight relative to the total weight of the alkane mixture. In some aspects, the n-hexadecane is about 0% by weight relative to the total weight of the alkane mixture.

In some aspects, the linear alkanes comprise about 0% to about 5% by weight of n-octadecane, about 1% to about 4% by weight of n-octadecane, or about 2% to about 3% by weight of n-octadecane relative to the total weight of the alkane mixture. In some aspects, the n-octadecane is about 0% by weight, about 1% by weight, about 2% by weight, about 3% by weight, about 4% by weight, or about 5% by weight relative to the total weight of the alkane mixture. In some aspects, the n-octadecane is about 0% by weight relative to the total weight of the alkane mixture.

In some aspects, the linear alkanes are of plant origin. In some aspects, the linear alkane of plant origin is n-decane. In some aspects, the linear alkane of plant origin is n-dodecane. In some aspects, the linear alkane of plant origin is n-tetradecane. In some aspects, the linear alkane of plant origin is n-hexadecane. In some aspects, the linear alkane of plant origin is n-octadecane.

In some aspects, the linear alkanes are free of alkanes with odd number carbon atoms. In some aspects, the linear alkane is n-decane. In some aspects, the linear alkane is n-dodecane. In some aspects, the linear alkane is n-tetradecane. In some aspects, the linear alkane is n-hexadecane. In some aspects, the linear alkane is n-octadecane.

In some aspects, the volatile alkane mixture comprises about 10% to about 90% by weight of branched C₁₀-C₁₄ alkanes, about 10% to about 85% by weight of branched C₁₀-C₁₄ alkanes, about 10% to about 80% by weight of branched C₁₀-C₁₄ alkanes, about 15% to about 75% by weight of branched C₁₀-C₁₄ alkanes, about 15% to about 70% by weight of branched C₁₀-C₁₄ alkanes, about 20% to about 65% by weight of branched C₁₀-C₁₄ alkanes, about 20% to about 60% by weight of branched C₁₀-C₁₄ alkanes, about 25% to about 55% by weight of branched C₁₀-C₁₄ alkanes, about 25% to about 50% by weight of branched C₁₀-C₁₄ alkanes, about 30% to about 45% by weight of branched C₁₀-C₁₄ alkanes, or about 30% to about 40% by weight of branched C₁₀-C₁₄ alkanes relative to the total weight of the alkane mixture.

### II. Crosslinked Elastomers

In some aspects, the person care compositions comprise one or more crosslinked elastomers. In some aspects, the crosslinked elastomer has particle sizes from about 1 micron to about 500 microns, about 20 micron to about 400 microns, about 30 micron to about 300 microns, about 50 micron to about 200 microns, or about 70 micron to about 150 microns. In some aspects, the crosslinked elastomer has particle sizes about 1 micron, about 50 microns, about 100 microns, about 150 microns, about 200 microns, about 250 microns, about 300 microns, about 350 microns, about 400 microns, about 450 microns, or about 500 microns.

In some aspects, the particles of the crosslinked elastomer are spherical. Examples of spherical crosslinked elastomer particles include but are not limited to elastomer particles with INCI INCI (International Nomenclature Cosmetic Ingredient) names Vinyl Dimethicone/Methicone Silsesquioxane Crosspolymer, Diphenyl Dimethicone/Vinyl Diphenyl Dimethicone/Silsesquioxane Crosspolymer, Polymethylsilsesquioxane, Dimethicone/Vinyl Dimethicone Crosspolymer, Dimethiconol/Silsesquioxane Copolymer and are available under commercial names Tospearl 2000B from Momentive Performance Materials, KSP-100, KSP-300 by Shinetsu siliocnes, DOWSIL™ EP-9801 Hydro Cosmetic Powder from Dow, Serasense SP-13 and Serasense SP-14 from KCC silicones.

In some aspects, the particles of the crosslinked elastomer are non-spherical.

In some aspects, the crosslinked elastomer is a crosslinked siloxane elastomer, a crosslinked polyurethane elastomer, a crosslinked polyester elastomer, or combinations thereof.

In some aspects, the crosslinked elastomer is a crosslinked siloxane elastomer. Examples of such elastomers include elastomers with INCI (International Nomenclature Cosmetic Ingredient) names dimethicone/vinyl dimethicone crosspolymers, Dimethiconol/Silsesquioxane Copolymer, Cetearyl Dimethicone Crosspolymer, C₃₀-45 Alkyl Cetearyl Dimethicone Crosspolymer, polysilicone-11 and sold under commercial names SFE839, silsoft silicone gel, Velvesil 125, Velvesil 034, Velvesil DM gel by Momentive Performance Materials, DOWSIL™ 9045 Silicone Elastomer Blend by Dow, Gransil EP-LS from grant industries.

In some aspects, the crosslinked elastomer is a crosslinked polyurethane elastomer. Examples of such elastomers are outlined in US20210059924A1.

In some aspects, the crosslinked elastomer is a crosslinked polyester elastomer or mixtures thereof. Examples of such elastomers include but are not limited to crosspolymer of sebacic acid and hydrogenated castor oil, crosspolymer of epoxidized soyabean oil and hydrogenated dimer acid and are outlined in US20220195178A1 and US20220194912A1.

In some aspects, the crosslinked elastomer is an emulsifying crosslinked elastomer. Examples of emulsifying crosslinked elastomers include but are not limited to elastomers with INCI names Dimethicone/Polyglycerin-3 Crosspolymer, Dimethicone/PEG-10/15 Crosspolymer, PEG-15/Lauryl Dimethicone Crosspolymer, PEG-12 Dimethicone/PPG-20 Crosspolymer and are commercially available under KSG 210, KSG 310, KSG 710 from Shinetsu Silicones, DOWSIL™ EL-7040 Hydro Elastomer Blend from Dow.

In some aspects, the crosslinked elastomer is a swellable crosslinked elastomer, a non-swellable crosslinked elastomer, or combinations thereof. In some aspects, the crosslinked elastomer is a swellable crosslinked elastomer.

In some aspects, the swellable crosslinked elastomer swells with a solvent to form a swollen crosslinked elastomer. In some aspects, the solvent is about 10% by weight, about 20% by weight, about 30% by weight, about 40% by weight, about 50% by weight, about 60% by weight, about 70% by weight, about 80% by weight, about 90% by weight, about 95% by weight, about 98% by weight, about 99% by weightof the swollen crosslinked elastomer.

In some aspects, the solvent is a volatile alkane mixture of the invention, a silicone solvent, a hydrocarbon solvent, an organic solvent, a fluorinated solvent, or combinations thereof. In some aspects, the solvent comprises about 0% to about 100% by weight of the volatile alkane mixture relative to the total weight of the solvent. In some aspects, the solvent comprises about 5% to about 95% by weight of the volatile alkane mixture, about 10% to about 90% by weight of the volatile alkane mixture, about 15% to about 85% by weight of the volatile alkane mixture, about 20% to about 80% by weight of the volatile alkane mixture, about 25% to about 75% by weight of the volatile alkane mixture, about 30% to about 70% by weight of the volatile alkane mixture, about 35% to about 65% by weight of the volatile alkane mixture, about 40% to about 60% by weight of the volatile alkane mixture, about 45% to about 55% by weight of the volatile alkane mixture, or about 50% to about 55% by weight of the volatile alkane mixture relative to the total weight of the solvent. In some aspects, the volatile alkane mixture is contained inside the swollen crosslinked elastomer.

In some aspects, the swollen crosslinked elastomer is a gel.

In some aspects, the swollen crosslinked elastomer has viscosity (25 °C) of about 10,000 cP to about 2,000,000 cP, about 20,000 cP to about 1,800,000 cP, about 40,000 cP to about 1,500,000 cP, about 50,000 cP to about 1,000,000 cP, or about 100,000 cP to about 500,000 cP.

### III. Personal Care Composition Ingredients

In some aspects, the personal care composition further comprises one or more of particulates, pigments, and dyes. In some aspects, the particulates, pigments and dyes are selected from the group consisting of mica, magnesium carbonate, calcium carbonate, magnesium silicate, aluminum magnesium silicate, silica, talc, aluminum starch octenylsuccinate, polymethylsilsesquioxane, nylon-12, starch particles including tapioca starch and rice starch, cellulose particles, cellulose acetate particles, synthetic dyes including azo dyes, lakes, carmines and botanicals, iron oxide, zinc oxide, titanium dioxide, boron nitride, red iron oxide, yellow iron oxide, black iron oxide, organo-modified hectorite clay, polystyrene powder, silk powder, crystalline cellulose, titanated mica, iron oxide titanated mica, bismuth oxychloride, pearl, pearl mica, interference pigments, and combinations thereof.

In some aspects, the personal care composition further comprises one or more organic sunscreen oils selected from the group consisting of avobenzone, octisalate, oxybenzone, homosalate, octocrylene, octyl methoxycinnamate, 2-ethoxyethyl p-methoxycinnamate, ethylhexyl salicylate, ethylhexyl methoxycinnamate, and combinations thereof.

In some aspects, the personal care composition further comprises a humectant selected from the group consisting of propylene glycol, dipropylene glycol, polypropylene glycol, polyethylene glycol, sorbitol, hydroxypropyl sorbitol, hexylene glycol, glycerin, 1,3-butylene glycol, 1,2,6-hexanetriol, ethoxylated glycerin, propoxylated glycerin, and combinations thereof.

In some aspects, the personal care composition further comprises one or more resins. In some aspects, the resin is selected from the group consisting of a silicone resin, a hydrocarbon resin, an organic resin, a fluorinated resin, and combinations thereof.

In some aspects, the resin is a silicone resin comprising monofunctional siloxane units (M units), difunctional siloxane units (D units), trifunctional siloxane units (T units), quattrofunctional siloxane units (Q units), or combinations thereof. In some aspects, the resin is a silicone resin comprising monofunctional siloxane units (M units) having alkyl and/or aryl substituents on silicone atoms, difunctional siloxane units (D units) having alkyl and/or aryl substituents on silicone atoms, trifunctional siloxane units (T units) having alkyl and/or aryl substituents on silicone atoms, quattrofunctional siloxane units (Q units) having alkyl and/or aryl substituents on silicone atoms, or combinations thereof. In some aspects, the resin is a silicone resin selected from the group consisting of MQ resin, MT resin, T-propyl resin, and combinations thereof.

In some aspects, the resin is an organic resin comprising homopolymers or co-polymers of vinyl polymers, acrylic polymers, methacrylic polymers, polyalkylenes, polyvinylpyrrolidones (PVP), vinylpyrrolidones (VP), or styrenic polymers, or combinations thereof.

In some aspects, the resin is an organic resin comprising polymeric gums, cellulose, cellulose derivatives, agar, pectin, gelatin, starch, starch derivatives, chitosan, chitosan derivatives, or combinations thereof. In some aspects, the polymeric gums are selected from the group consisting of alginates, carageenans, gum acacia, gum arabic, gum ghatti, gum karaya, gum tragacanth, guar gum, guar hydroxypropyltrimonium chloride, xanthan gum, gellan gum, dammar gum, olibanum gum, and combinations thereof. In some aspects, the cellulose derivatives are selected from the group consisting of sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropylcellulose, methyl cellulose, hydroxypropylmethyl cellulose, microcrystalline cellulose, and combinations thereof. In some aspects, the chitosan derivative is hydroxyethyl chitosan.

In some aspects, the resin is a film former.

In some aspects, the personal care composition further comprises a silicone wax, an organic wax, or combinations thereof. In some aspects, the silicone wax is selected from the group consisting of C₃₀-C₄₅ alkyl dimethicone, C₃₀-C₄₅ alkyl cetearyl dimethicone crosspolymer, and combinations thereof. In some aspects, the organic wax is selected from the group consisting of beeswax, ozokerite wax, carnauba wax, shea butter, stearic acid, cetyl alcohol, and combinations thereof.

In some aspects, the personal care composition further comprises an aqueous phase selected from the group consisting of water, glycerin, butylene glycol, propanediol, xanthan gum, steareth 2, steareth 21, sodium polyacrylate, disodium EDTA, hydroxyethyl urea, a cationic emulsifier, an anionic emulsifier, a non-ionic emulsifier, sodium chloride, and combinations thereof.

In some aspects, the personal care composition further comprises one or more non-volatile oils selected from the group consisting of a silicone oil, a hydrocarbon oil, a fluorinated oil, an organic oil, and combinations thereof.

In some aspects, the non-volatile oil is a silicone gum selected from the group consisting of polyalkylsiloxane, polyarylsiloxane, polyalkylsilanol, and combinations thereof.

In some aspects, the non-volatile oil is selected from the group consisting of an amino modified silicone, a polyether modified silicone, a phenyl modified silicone, and combinations thereof.

In some aspects, the non-volatile oil is a phenyl modified silicone selected from the group consisting of phenyltrimethicone, triphenyl dimethylvinyl disiloxane, phenylpropyldimethylsiloxysilicate, and combinations thereof.

In some aspects, the non-volatile oil is an organic oil selected from the group consisting of a C₈-C₄₀ fatty alcohol, an ester of a C₇-C₃₉ fatty acid, an ester of a C₈-C₄₀ fatty alcohol, polyisobutene, a wax, a plant oil including coconut oil, almond oil and avocado oil, a mineral oil, a synthetic oil, and combinations thereof. In some aspects, the organic oil is a C₈-C₄₀ fatty alcohol, a C₁₀-C₄₀ fatty alcohol, a C₁₅-C₃₅ fatty alcohol, a C₂₀-C₃₀ fatty alcohol, or a C₂₅-C₃₀ fatty alcohol. In some aspects, the organic oil is an ester of a C₇-C₃₉ fatty acid, an ester of a C₁₀-C₃₅ fatty acid, an ester of a C₁₅-C₃₀ fatty acid, or an ester of a C₂₀-C₂₅ fatty acid. In some aspects, the organic oil is an ester of a C₈-C₄₀ fatty alcohol, an ester of a C₁₀-C₄₀ fatty alcohol, an ester of a C₁₅-C₃₅ fatty alcohol, an ester of a C₂₀-C₃₅ fatty alcohol, or an ester of a C₂₅-C₃₀ fatty alcohol.

In some aspects, wherein the non-volatile oil is selected from the group consisting of a conditioning agent, an emollient, a sunscreen oil, a natural oil, and combinations thereof.

In some aspects, the personal care composition further comprises one or more preservatives. In some aspects, the preservative is phenoxyethanol, ethylhexylglycerin, or combinations thereof.

In some aspects, the personal care composition further comprises one or more fragrances.

In some aspects, the personal care composition is a liquid, an emulsion, a lotion, a paste, a cream, an aerosol spray, a solid stick, a gel, a biphasic liquid, or a multi phasic liquid.

In some aspects, the personal care composition is a deodorant, an antiperspirant, a skin cream, a facial cream, a hair shampoo, a hair conditioner, a mousse, a hair styling gel, a hair spray, a protective cream, a lipstick, a lip color, lip gloss, sunscreen, after sun lotion, sun spray, a primer, a facial foundations, blushes, makeup, mascara, a skin care lotion, a moisturizer, a facial treatment, eye serum, eye cream, a personal cleanser, a facial cleanser, a bath oil, a perfume, a shaving cream, a pre-shave lotion, an after-shave lotion, a cologne, a sachet, loose facial powder, compact powder, eye shadow, or a sunscreen.

### Example 1

### Preparation of Alkane Mixture Examples 1-3 and Comparative Examples 1-2

The mixture of linear alkane of the invention is produced as outlined in EP2417199A1. Linear alkanes and branched C₁₀-C₁₂ alkane were combined and stirred until a homogeneous solution was formed. Table 1 lists the ingredients of alkane mixture examples 1-3 and comparative examples 1-2.

TABLE 1

| | | | | Comparative | Comparative |
|---|---|---|---|---|---|
| Composition Wt. % | Example 1 | Example 2 | Example 3 | Example 1 | Example 2 |
| Linear alkane (C₁₀) | 2.8 | 0 | 0.7 | | |
| Linear alkane (C₁₂) | 64.1 | 69.5 | 61.7 | | |
| Linear alkane (C₁₄) | 3.4 | 1.3 | 2.1 | | |
| Linear alkane (C₁₆- | 0 | 2.4 | 4.8 | | |
| C₁₈) | | | | | |
| Branched alkane | 29.7 | 26.8 | 30.7 | 100 | |
| (C₁₀-C₁₂) | | | | | |
| Cyclopentasiloxane | | | | | 100 |

### Example 2

### Preparation of Personal Care Formulation 1

Table 2 lists the ingredients of Personal Care Formulation 1 (gel). The procedure for preparing Personal Care Formulation 1 is described as following. Alkane mixture example 3 and dimethiconol/silsesquioxane copolymer were combined and stirred at 3000 rpm until a homogeneous solution was formed. Comparative example 1 and dimethiconol/silsesquioxane copolymer were combined and stirred at 3000 rpm until a homogeneous solution was formed. Comparative example 2 and dimethiconol/silsesquioxane copolymer were combined and stirred at 3000 rpm until a homogeneous solution was formed.

Table 3 lists the wettability, processibility and tack attributes of Formulation Example 1a-1c. Formulation Example 1a comprising alkane mixture example 3 shows better wettability, shorter processing time and reduced tack as compared to benchmark Formulation Examples 1b and 1c.

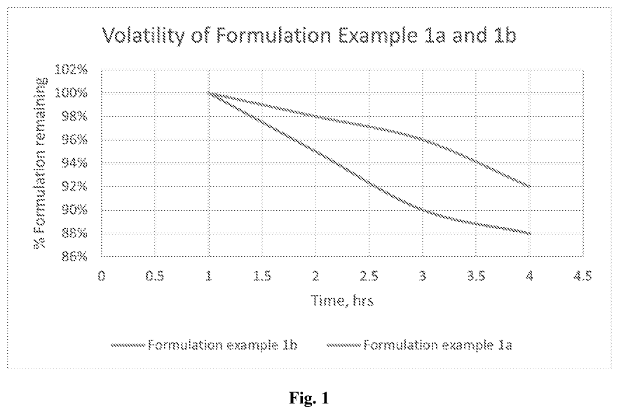

FIG. 1 describes the volatility of Formulation Examples 1a and 1b. In FIG. 1, Formulation Example 1b shows higher volatility as compared to Formulation Example 1a. Lower volatility of Formulation 1a leads to higher rub time, which provides higher cushioning, yet drier feel as compared to Formulation 1b. Formulation Example 1a exhibits lower tack and thus exhibits light non-tacky feel as compared to Formulation Examples 1b and 1c.

TABLE 2

| | Formulation | Formulation | Formulation |
|---|---|---|---|
| Ingredient | Example 1a | Example 1b | Example 1c |
| Alkane Mixture Example 3 | 75% | | |
| Comparative Example 1 | | 75% | |
| Comparative Example 2 | | | 75% |
| Crosslinked polymer | 25% | 25% | 25% |
| (Dimethiconol/Silsesquioxane | | | |
| Copolymer) | | | |

TABLE 3

| | Formulation | Formulation | Formulation |
|---|---|---|---|
| Attribute | Example 1a | Example 1b | Example 1c |
| Wettability | ***** | ** | ** |
| Processibility | ***** | *** | ** |
| Tack, N¹ | 24.86 | 60.53 | 60.76 |

Tack is measured by rheometer MCR 301 (Anton Paar, Austria), probe PP25, v=0.5 mm/sec.

### Example 3

### Preparation of Personal Care Formulation 2

Table 4 lists the ingredients of Personal Care Formulation 2 (anti-ageing facial serum). The procedure for preparing Personal Care Formulation 2 is described as following. Phase A ingredients were combined and stirred at room temperature until a homogeneous solution was formed. Formulation Example 2a containing alkane mixture example 1 shows better wettability, shorter processing time and an enhanced sensory profile including non-greasy and non-tacky feel as compared to benchmark Formulation Examples 2b and 2c.

TABLE 4

| | | | Formulation | Formulation | Formulation |
|---|---|---|---|---|---|
| Phase | Ingredient | INCI Name | Example 2a | Example 2b | Example 2c |
| A | PDMS-5 | Poly(dimethylsiloxane) | 63% | 63% | 63% |
| | Almond Oil | Prunus | 0.5% | 0.5% | 0.5% |
| | | Amygdalus | | | |
| | | Dulcis (Sweet | | | |
| | | Almond) Oil | | | |
| | Dimethiconol/ | Dimethiconol/ | 22% | 22% | 22% |
| | Silsesquioxane | Silsesquioxane | | | |
| | Copolymer | Copolymer | | | |
| | Alkane | Alkane | 10% | | |
| | Mixture | Mixture | | | |
| | Example 1 | Example 1 | | | |
| | Comparative | Isododecane | | 10% | |
| | Example 1 | | | | |
| | Comparative | Cyclopentasiloxane | | | 10% |
| | Example 2 | | | | |
| | GTCC | Caprylic/Capric | 4% | 4% | 4% |
| | | Triglyceride | | | |
| | Argan Oil | Argania | 0.5% | 0.5% | 0.5% |
| | | Spinosa Kernel | | | |
| | | Oil | | | |

### Example 4

### Preparation of Personal Care Formulation 3

Table 5 lists the ingredients of Personal Care Formulation 3 (mineral sunscreen). The procedure for preparing Personal Care Formulation 3 is described as following. Mix Phase A ingredients and homogenize at 10,000 rpm for 1 minute until uniform. Mix Phase B ingredients in a separate vessel. Slowly add Phase B into Phase A and mix to emulsify. Homogenize at 5,000 rpm for 5 minutes.

TABLE 5

| | Trade Name | INCI Name | Wt % |
|---|---|---|---|
| Phase A | Harmonie ™ Mesh Emulsifier | (Caprylic/Capric) Triglyceride (and) PEG/PPG- | 2.00 |
| | | 20/15 Dimethicone | |
| | Silsoft 034 Fluid | Caprylyl methicone | 5.00 |
| | Alkane Mixture Example 2 | Alkane Mixture Example 2 | 20.00 |
| | Serasense SF5 (5 cst) | Dimethicone | 10.00 |
| | ZnO—C (Non-nano) | Zinc Oxide | 10.00 |
| | Harmonie Luxe 4 powder | Silica | 4.00 |
| | Velvesil DM LC | Dimethicone | 4.00 |
| | | (and) Cetearyl Dimethicone Crosspolymer | |
| | SS4267 | Dimethicone (and)Trimethylsiloxysilicate | 5.70 |
| Phase B | Water | Water | 35.40 |
| | Butylene Glycol | Butylene Glycol | 2.00 |
| | Sodium Chloride | Sodium Chloride | 0.40 |
| | Polysorbate 20 | Polysorbate 20 | 0.90 |
| | Euxhyl 9010 | Phenoxyethanol | 0.60 |

### Example 5

### Preparation of Personal Care Formulation 4

Table 6 lists the ingredients of Personal Care Formulation 4 (mineral sunscreen). The procedure for preparing Personal Care Formulation 4 is described as following. Mix Phase A ingredients and homogenize at 10,000 rpm for 1 minute until uniform. Mix Phase B ingredients in a separate vessel. Slowly add Phase B into Phase A and mix to emulsify. Homogenize at 5,000 rpm for 5 minutes.

TABLE 6

| | Trade Name | INCI Name | Wt % |
|---|---|---|---|
| Phase A | Harmonie ™ Mesh Emulsifier | (Caprylic/Capric) Triglyceride (and) PEG/PPG- | 2.00 |
| | | 20/15 Dimethicone | |
| | Silsoft 034 Fluid | Caprylyl methicone | 5.00 |
| | Alkane Mixture Example 3 | Alkane Mixture Example 3 | 20.00 |
| | Dermol 99 | Isononyl Isononanoate | 10.00 |
| | ZnO—C (Non-nano) | Zinc Oxide | 10.00 |
| | Harmonie Luxe 4 powder | Silica | 4.00 |
| | Sebcic acid/hydrogenated castor oil | Sebcic acid/hydrogenated castor oil | 4.00 |
| | crosspolymer | crosspolymer | |
| | SS4267 | Dimethicone (and)Trimethylsiloxysilicate | 5.70 |
| Phase B | Water | Water | 35.40 |
| | Glycerin | Glycerin | 2.00 |
| | Sodium Chloride | Sodium Chloride | 0.40 |
| | Polysorbate 20 | Polysorbate 20 | 0.90 |
| | Euxhyl 9010 | Phenoxyethanol | 0.60 |

### Example 6

### Preparation of Personal Care Formulation 5

Table 7 lists the ingredients of Personal Care Formulation 5 (body lotion). The procedure for preparing Personal Care Formulation 5 is described as following. Combine phase (A) ingredients and homogenize for 5 minutes at 2000 rpm (cowles mixer). Add the additional phase B to the previous phase and homogenize.

TABLE 7

| | Trade Name | INCI Name | Wt % |
|---|---|---|---|
| Phase A | Aqua | Aqua | Up to |
| | | | 100 |
| | Silsoft* EAU microgel | Polysilicone-34 (and) Isononyl isonanoate | 2.6 |
| | | (and) Water | |
| | Velvesil E Gel PMF emulsion | Dimethicone/Vinyl Dimethicone Crosspolymer | 2.0 |
| | | (and) Dimethicone (and) Isohexadecane (and) | |
| | | Cetearyl Methicone | |
| Phase B | Velvesil* DM gel | Dimethicone (and) Cetearyl Dimethicone | 5.0 |
| | | Crosspolymer | |
| | Silsoft* ETS fluid | Ethyl Trisiloxane | 6.0 |
| | Dermol 99 | Isononyl Isononanoate | 3.0 |
| | Alkane Mixture Example 3 | Alkane Mixture Example 3 | 5.0 |
| | Tospearl 2000B | Polymethylsilsesquioxane | 1.0 |

1. A personal care composition comprising (a) a volatile alkane mixture comprising about 10% to about 90% by weight of linear alkanes having 9 to 18 carbon atoms and about 10% to about 90% by weight of branched alkanes having 10 to 14 carbon atoms relative to the total weight of the alkane mixture; and (b) one or more crosslinked elastomers.

2. The personal care composition of claim 1, wherein the volatile alkane mixture comprises about 30% to about 85% by weight of linear alkanes having 9 to 18 carbon atoms relative to the total weight of the alkane mixture.

3. The personal care composition of claim 1, wherein the volatile alkane mixture comprises about 50% to about 75% by weight of linear alkanes having 9 to 18 carbon atoms relative to the total weight of the alkane mixture.

4. The personal care composition of any one of claims 1-3, wherein the linear alkanes comprise about 0% to about 5% by weight of n-decane, about 50% to about 80% by weight of n-dodecane, about 1% to about 5% by weight of n-tetradecane, and about 0% to about 5% by weight of n-hexadecane or n-octadecane relative to the total weight of the alkane mixture.

5. The personal care composition of any one of claims 1-4, wherein the linear alkanes are of plant origin.

6. The personal care composition of any one of claims 1-5, wherein the linear alkanes are free of alkanes with odd number carbon atoms.

7. The personal care composition of any one of claims 1-6, wherein the crosslinked elastomer has particle sizes from about 1 micron to about 500 microns.

8. The personal care composition of any one of claims 1-7, wherein the crosslinked elastomer is a crosslinked siloxane elastomer, a crosslinked polyurethane elastomer, a crosslinked polyester elastomer, or combinations thereof.

9. The personal care composition of any one of claims 1-8, wherein the crosslinked elastomer is an emulsifying crosslinked elastomer.

10. The personal care composition of any one of claims 1-9, wherein the crosslinked elastomer is a swellable crosslinked elastomer, a non-swellable crosslinked elastomer, or combinations thereof.

11. The personal care composition of claim 10, wherein the swellable crosslinked elastomer swells with a solvent to form a swollen crosslinked elastomer.

12. The personal care composition of claim 11, wherein the solvent is about 10% or more by weight relative to the weight of the swollen crosslinked elastomer.

13. The personal care composition of claim 11 or 12, wherein the solvent is a volatile alkane mixture, a silicone solvent, a hydrocarbon solvent, an organic solvent, a fluorinated solvent, or combinations thereof.

14. The personal care composition of any one of claims 11-13, wherein the solvent comprises the volatile alkane mixture from about 0% to about 100% by weight of the total weight of the solvent.

15. The personal care composition of any one of claims 11-14, wherein the swollen crosslinked elastomer has viscosity of about 10,000 cP to about 2,000,000 cP at 25 °C.

16. The personal care composition of any one of claims 11-15, wherein the swollen crosslinked elastomer is a gel.

17. The personal care composition of any one of claims 1-16, wherein the personal care composition further comprises one or more of particulates, pigments, and dyes.

18. The personal care composition of claim 17, wherein the particulates and pigments are selected from the group consisting of mica, magnesium carbonate, calcium carbonate, magnesium silicate, aluminum magnesium silicate, silica, talc, aluminum starch octenylsuccinate, polymethylsilsesquioxane, nylon-12, starch particles including tapioca starch and rice starch, cellulose particles, cellulose acetate particles, titanium dioxide, zinc oxide, boron nitride, red iron oxide, yellow iron oxide, black iron oxide, organo-modified hectorite clay, polystyrene powder, silk powder, crystalline cellulose, titanated mica, iron oxide titanated mica, bismuth oxychloride, pearl, pearl mica, interference pigments, and combinations thereof.

19. The personal care composition of any one of claims 1-18, wherein the personal care composition further comprises one or more organic sunscreen oils selected from the group consisting of avobenzone, octisalate, oxybenzone, homosalate, octocrylene, octyl methoxycinnamate, 2-ethoxyethyl p-methoxycinnamate, ethylhexyl salicylate, ethylhexyl methoxycinnamate, and combinations thereof.

20. The personal care composition of any one of claims 1-19, wherein the personal care composition further comprises a humectant selected from the group consisting of propylene glycol, dipropylene glycol, polypropylene glycol, polyethylene glycol, sorbitol, hydroxypropyl sorbitol, hexylene glycol, glycerin, 1,3-butylene glycol, 1,2,6-hexanetriol, ethoxylated glycerin, propoxylated glycerin, and combinations thereof.

21. The personal care composition of any one of claims 1-20, wherein the personal care composition further comprises one or more resins.

22. The personal care composition of claim 21, wherein the one or more resin is a silicone resin selected from the group consisting of MQ resin, MT resin, T-propyl resin, and combinations thereof.

23. The personal care composition of any one of claims 1-22, wherein the personal care composition is a liquid, an emulsion, a lotion, a paste, a cream, an aerosol spray, a solid stick, a gel, a biphasic liquid, or a multi phasic liquid.

24. The personal care composition of any one of claims 1-23, wherein the personal care composition is a deodorant, an antiperspirant, a skin cream, a facial cream, a hair shampoo, a hair conditioner, a mousse, a hair styling gel, a hair spray, a protective cream, a lipstick, a lip color, lip gloss, sunscreen, after sun lotion, sun spray, a primer, a facial foundations, blushes, makeup, mascara, a skin care lotion, a moisturizer, a facial treatment, eye serum, eye cream, a personal cleanser, a facial cleanser, a bath oil, a perfume, a shaving cream, a pre-shave lotion, an after-shave lotion, a cologne, a sachet, loose facial powder, compact powder, eye shadow, or a sunscreen.
